(19) United States
(12) Patent Application Publication
(10) Pub. No.: US 2026/0174397 A1
(43) Pub. Date: Jun. 25, 2026

(54) SYSTEMS AND METHODS FOR BRAIN SOURCE LOCALIZATION

(71) Applicant: Duke University, Durham, NC (US)

(72) Inventors: Warren Grill, Durham, NC (US); Brandon Thio, Durham, NC (US)

(21) Appl. No.: 19/126,832

(22) PCT Filed: Nov. 2, 2023
(86) PCT No.: PCT/US2023/078512

§ 371 Date: May 2, 2025

Related U.S. Application Data
(60) Provisional application No. 63/422,172, filed on Nov. 3, 2022.

(51) Int. Cl. A61B 5/00; A61B 5/374

(52) U.S. Cl. CPC A61B 5/7275; A61B 5/374; A61B 5/4094; A61B 5/7264; A61B 5/7267

(57) ABSTRACT

The present disclosure provides systems and methods for brain source localization. In particular, the present disclosure provides a novel source reconstruction method (termed “TEmporally Dependent Iterative Expansion” or TEDIE), that accurately reconstructs propagating and expanding neural sources over time. The systems and method described herein can be used as a clinical tool to enhance epileptogenic zone localization and improve surgical outcomes.

### RELATED APPLICATIONS

This application claims priority to and the benefit of U.S. Provisional Patent Application No. 63/422,172 filed Nov. 3, 2022, which is incorporated herein by reference in its entirety for all purposes.

### GOVERNMENT FUNDING

This invention was made with Government support under Federal Grant No. F31NS124094 awarded by the National Institute of Neurological Disorders & Stroke (NIH/NINDS) and Federal Grant No. TR002553 awarded by the National Institutes of Health (Clinical and Translational Science Award). The Federal Government has certain rights to the invention.

### FIELD

The present disclosure provides systems and methods for brain source localization. In particular, the present disclosure provides a novel source reconstruction method (termed “TEmporally Dependent Iterative Expansion” or TEDIE), that accurately reconstructs propagating and expanding neural sources over time. The systems and method described herein can be used as a clinical tool to enhance epileptogenic zone localization and improve surgical outcomes.

### BACKGROUND

Successful surgical outcomes for patients with epilepsy require proper identification of the brain tissues where seizures originate. Despite the technological advances in surgical planning and epilepsy monitoring, seizure-freedom rates from resection surgery have remained at ˜60%. In part, seizure-freedom rates have not improved because the same analysis tools to delineate the epileptogenic zone (EZ), the minimum amount of brain tissue that needs to be removed to achieve seizure-freedom, have been used for several decades. Clinical identification of the EZ is accomplished using intracranial stereo-EEG, whereby ˜5-30 wires, each with 4-18 recording contacts, are implanted, through small drill holes, into widespread regions of the brain. The contacts record brain activity over periods lasting 5-20 days, and simultaneous video monitoring is used to identify periods of clinical seizure activity. Epilepsy neurologists then manually examine the recordings to try to identify the EZ.

However, EZ localization is challenging because it requires examining hundreds of simultaneous recordings, identifying the few electrode contacts that initially register epileptic neural activity, and then moving back to the patient's anatomy to create a surgical plan. Additionally, electrodes that capture seizure initiation may not exhibit signals that are readily perceptible by human eye, and patients may present with multiple types of seizures originating from different locations. Although epilepsy neurologists spend ˜1-2 hours to examine each 24 hours of recordings, >40% of patients do not progress to surgery (i.e., are not offered therapy).

Recently, computational source localization algorithms have been implemented on stereo-EEG data to localize interictal spikes and seizures to inform EZ localization. However, all of the algorithms implemented on stereo-EEG data were originally developed to localize the neural sources from extracranial EEG. Compared to extracranial EEG, stereo-EEG electrodes are much closer to the active neurons and thus record much higher SNR and potentially more informative signals. Therefore, new algorithms that can take advantage of the higher quality signals need to be developed. Additionally, the algorithms previously applied to stereo-EEG data are static and determined the location of neural activity for a single time point. However, dynamic source localization from extracranial EEG greatly outperforms static source localization and can inform not just the location but also the size of neural sources.

While dynamic source reconstruction algorithms localize stationary neural sources very well from the temporal dynamics of neural recordings, these algorithms do not account for dynamic changes in the size and location (propagation) of seizure activity. The neural tissues generating the recorded signals at one point in time are not necessarily the same as the tissues at the next point in time. However, there is information from localization at one point in time that can inform the localization at subsequent points in time.

### SUMMARY

Embodiments of the present disclosure include a method for reconstructing propagating neural activity over time. In accordance with these embodiments, the method includes determining location and size estimates for a plurality of active neural sources based on neural activity at a plurality of time points; clustering adjacent populations of neural sources together to generate time-dependent clusters of neural sources; determining a temporal average of the location and size of each cluster; optimizing the location and size of each cluster by minimizing error between reconstructed and recorded signals; and generating a neural source likelihood map corresponding to likelihood that an active neural source is present at a specific location and at a specific time. In some embodiments, neural activity is recorded using a plurality of electrodes (e.g., sEEG, EEG, or HD-EEG).

In some embodiments, the method further comprises repeating the step of determining a temporal average of the location and size of each cluster, and/or repeating the step of optimizing the location and size of each cluster by minimizing error between reconstructed and recorded signals.

In some embodiments, the method further comprises minimizing error between a reconstructed and average location, and/or minimizing error between a reconstructed and average size.

In some embodiments, the method minimizes distance between neural sources in a cluster of neural sources across time. In some embodiments, the method minimizes changes in size between neural sources in a cluster of neural sources across time.

In some embodiments, the neural activity comprises patient-specific interictal spike and/or seizure recordings. In some embodiments, the active neural source corresponds to a seizure onset zone.

In some embodiments, the method further comprises projecting the likelihood map onto a map of a patient's brain anatomy.

In some embodiments, the method is capable of diagnosing a patient as being seizure-free or not seizure-free. In some embodiments, the method is capable of identifying an active neural source as a target for surgical intervention.

In some embodiments, determining the location and size estimates comprises obtaining recordings from a plurality of electrodes that are compatible with electroencephalography (EEG), stereo-electroencephalography (sEEG), and/or magnetoencephalography (MEG).

In some embodiments, the method identifies an active neural source with a Jaccard index of at least about 0.5. In some embodiments, the method identifies an active neural source with a mapped percentage of at least about 55%. In some embodiments, the method identifies an active neural source with a center of mass localization error of no more than about 6.0 mm.

Embodiments of the present disclosure also include a method for determining probability of post-surgical seizure freedom in a patient. In accordance with these embodiments, the method includes performing epileptogenic zone localization in the patient using any of the methods described herein; determining average epileptogenic zone location based on center of mass of each epileptogenic zone; determining distance between the average epileptogenic zone location and the center of mass of each epileptogenic zone (dCOM value); and comparing the average epileptogenic zones locations from patients post-surgery to the patient to determine the probability of post-surgical seizure freedom in the patient.

In some embodiments, the method further comprises determining a confidence interval associated with the probability of post-surgical seizure freedom in the patient based on historical data of average epileptogenic zones locations in a plurality of post-surgical patients.

In some embodiments, performing epileptogenic zone localization comprises determining patient-specific interictal spike and/or seizure recordings.

In some embodiments, the method further comprises compiling a plurality of average epileptogenic zone locations from patients who are and are not seizure-free into a database.

In some embodiments, the method further comprises creating a classification system that outputs a score indicating whether the patient will or will not benefit from surgical intervention. In some embodiments, the classification system comprises assessing an Engel score and/or an ILAE score.

Embodiments of the present disclosure also include a method for determining probability of post-surgical seizure freedom in a patient undergoing mesial temporal ablation therapy. In accordance with these embodiments, the method includes performing epileptogenic zone localization in the patient using any of the methods described herein; determining the proportion of the patient's epileptogenic zone location originating in the patient's neocortical anterior temporal lobe; and comparing the proportion of epileptogenic zone locations comprising neocortical anterior temporal lobe onset from patients post-surgery to the patient to determine probability of post-surgical seizure freedom in the patient.

In some embodiments, the method further comprises determining a confidence interval associated with the probability of post-surgical seizure freedom in the patient based on historical data of average epileptogenic zones locations in a plurality of post-surgical patients.

In some embodiments, performing epileptogenic zone localization comprises determining patient-specific interictal spike and/or seizure recordings.

In some embodiments, the method further comprises compiling a plurality of average epileptogenic zone locations from patients who are and are not seizure-free into a database.

In some embodiments, the method further comprises creating a classification system that outputs a score indicating whether the patient will or will not benefit from surgical intervention. In some embodiments, the classification system comprises assessing an Engel score and/or an ILAE score.

### BRIEF DESCRIPTION OF THE DRAWINGS

The accompanying figures are provided by way of illustration and not by way of limitation.

FIG. 1: Method for reconstructing neural sources with TEDIE. TEDIE uses recordings of seizure activity from stereo-EEG as inputs. TEDIE generates initial estimates of source size and location at each time point using best-fit dipole location and spatial extent. Each estimate source is temporally linked with other estimated sources that are within 4 cm of each other. An example of the temporal linking of sources close to the red source in time is shown. A Gaussian filter is applied to the source to find the temporally averaged source locations and sizes. The sources are optimized such that the error between the reconstructed and recorded signals are minimized and the difference between the current and averaged source estimates are minimized. Temporal averaging and source optimization are iteratively conducted until the simulation converges across iterations after which a source likelihood map is generated to provide the likelihood that a source is present at each cortical location within a specified window of time.

FIGS. 2A-2F: Performance of TEDIE localizing synthetic EZs compared to other previously developed source localization algorithms. A) Stationary and constant size source, B) propagating and constant size source, C) stationary and growing source, and D) propagating and growing source reconstructed by TEDIE, and other previously developed source localization algorithms sLORETA and IRES. E) Jaccard Index (Intersection divided by the union of ground truth and reconstructed sources: 1 is perfect overlap and 0 is disjoint sources) and F) localization error between center of mass for ground truth and reconstructed sources for synthetic sources with mean propagation speed, mean growth rate, and 20 dB SNR. TEDIE outperformed sLORETA and IRES using both metrics of source overlap with ground truth.

FIGS. 3A-3F: Performance of TEDIE reconstructing seizure onset zone. A) Seizure onset times were identified by including 100 timepoints where the maximum contact voltage exceeded a noise floor (3*standard deviation). B) Example of concordant seizure onset zone reconstruction and resected electrode contacts (yellow dots) for a seizure-free patient. C) Example of discordant seizure onset zone reconstruction and resected electrode contacts (yellow dots) for a patient with continuing seizures. D) Distance between the maximum likelihood source location and nearest resected electrode contacts for seizure-free (Engel 1) and not seizure-free patients (Engel 2-4). E) Distance between the furthest point in the reconstructed source and the resected electrode contacts for seizure-free (Engel 1) and not seizure-free patients (Engel 2-4). Subdividing by temporal lobe (purple) and extratemporal lobe (yellow) epilepsies show large differences between seizure-free and not seizure-free distance for extratemporal lobe epilepsy. F) Receiver operator curves of a classifier based on localization error for each of the four datasets. *p<0.05 from ANOVA and post hoc t tests.

FIGS. 4A-4F: Comparison of performance of TEDIE, sLORETA, and IRES in reconstructing seizure onset zones. Examples of A) TEDIE, B) sLORETA, and C) IRES reconstructing the same seizure onset zone where the yellow dot represents the electrode contact, projected to the brain surface, which was which was within the ablated region. D) Comparison of the distances between the maximum likelihood location in the reconstructions and the nearest electrode contact within the resected brain region for TEDIE (green), sLORETA (orange), and IRES (purple) for seizure-free and not seizure-free patients. E) Comparison of spatial diffusivity of the reconstructed seizure onset zones for TEDIE, sLORETA, and IRES. F) Comparison of temporal consistency of reconstructions throughout the time course of a seizure for TEDIE, sLORETA, and IRES. In all panels, each data point represents the median metric value for each patient. *p<0.05 from an ANOVA and post hoc t-tests.

FIGS. 5A-5E: Consistency of reconstructed seizure onset zones for Engel 1 and Engel 2-4 patients. A) Consistent seizure onset zones of two seizures from the same seizure-free patient after ATL resection. B) Inconsistent seizure onset zones of three seizures from the same not seizure-free patient after an MFL ablation. C) Mean distance between the average maximum likelihood source location across seizures and the maximum likelihood source location for each seizure for seizure-free and not seizure-free patients (Blue). Subdividing by temporal lobe (purple) and extratemporal lobe (yellow) epilepsies showed large differences between seizure-free and not seizure-free distance for extratemporal lobe epilepsy. D) Receiver operator curve of a classifier based on seizure onset zone consistency for each of the three datasets. E) AUROC of classifiers trained on seizure onset zone consistency when different numbers of seizures were used to calculate consistency. Error bars are 95% confidence intervals found by bootstrapping the AUROC with 1000 trials. *p<0.05 from ANOVA and posthoc t-tests.

FIGS. 6A-6D: Comparison of TEDIE reconstructions based on the outcomes of different temporal lobe surgical therapies. A) Example of a MTL seizure onset zone for a seizure-free patient who received a right MTL ablation. B) Example of an ATL seizure onset zone for a patient who was not seizure-free after a left MTL ablation. C) Example of an ATL and MTL seizure onset zone for a seizure-free patient who received an ATL resection. D) Percentage of seizures with ATL sources for seizure-free patients after a MTL ablation, not seizure-free patients after a MTL ablation, and seizure-free patients after an ATL resection.

FIGS. 7A-7C: Differences in TEDIE reconstructions for anisotropic and isotropic head models reconstructing synthetic neural sources generated by high mesh density anisotropic head models. A) Jaccard Index, B) % mapped, and C) center of mass localization error for anisotropic and isotropic head models along with the differences between the model types.

FIGS. 8A-8C: TEDIE's hyperparameter selection using 75:25 cross validation. A) Jaccard Index, B) % mapped, and C) center of mass localization error show the same best performing hyperparameters on the training data (α=0.1 and λ=100). Performance on the testing data confirmed the selection of hyperparameters.

FIGS. 9A-9L: Sensitivity of (A-C, G-I) AUROC and (D-F, J-L) maximum accuracy on (A-F) cutoff threshold and (G-L) time offset for the linear classifiers trained on max distance of the TEDIE reconstruction to the nearest resected electrode contacts. Classifiers trained on (A,D,G,J) all patient data, (B, E, H, K) temporal lobe patient data, and (C, F, I, L) extratemporal lobe patient data are shown.

FIG. 10: Sensitivity of detection of ATL sources for Engel 1 MTL ablation, Engel 2-4 MTL ablation, and Engel 1 ATL resection cases to changes in seizure onset window size and TEDIE reconstruction threshold.

FIG. 11: Sensitivity of detection of ATL sources for Engel 1 MTL ablation, Engel 2-4 MTL ablation, and Engel 1 ATL resection cases to changes in seizure onset window size and TEDIE reconstruction threshold. (The Sylvian fissure and superior temporal gyrus were omitted from the definition of ATL, which drives multiple thresholds (25-99%) and time windows (4-6 s) towards significance (p<0.1)). The single MTL patient that this change affects also had an ablation that was located <1.5 cm from the center of their seizure onset for multiple seizures.

FIG. 12: New targets for therapy predicted by TEDIE. Each visualization is labeled by patient ID and surgical intervention followed by TEDIE predicted target underneath.

FIG. 13: Jaccard index for the 27 different synthetic source types (3 propagation speeds, 3 growth rates, and 3 SNRs) and 4 different algorithms: TEDIE, sLORETA, IRES, and IE (Initial source estimation of TEDIE). Each data point is the median Jaccard index over time for a single source type and patient.

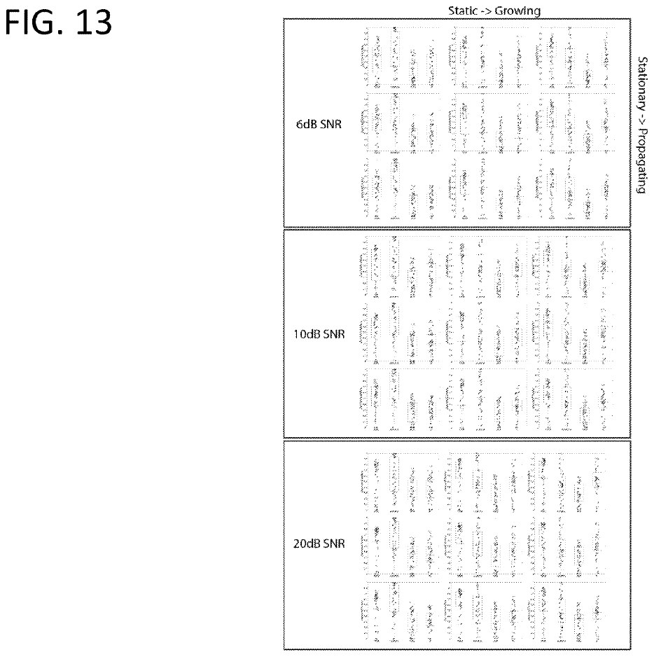

FIG. 14: Percent mapped for the 27 different synthetic source types (3 propagation speeds, 3 growth rates, and 3 SNRs) and 4 different algorithms: TEDIE, sLORETA, IRES, and IE (Initial source estimation of TEDIE). Each data point is the median % mapped over time for a single source type and patient.

FIG. 15: Center of mass localization error for the 27 different synthetic source types (3 propagation speeds, 3 growth rates, and 3 SNRs) and 4 different algorithms: TEDIE, sLORETA, IRES, and IE (Initial source estimation of TEDIE). Each data point is the median center of mass localization error over time for a single source type and patient.

FIG. 16: Sensitivity of linear classifier performance to chosen time for source localization with sLORETA and IRES. Red dots correspond to the peak time. Classifiers are built using the best-resected distance.

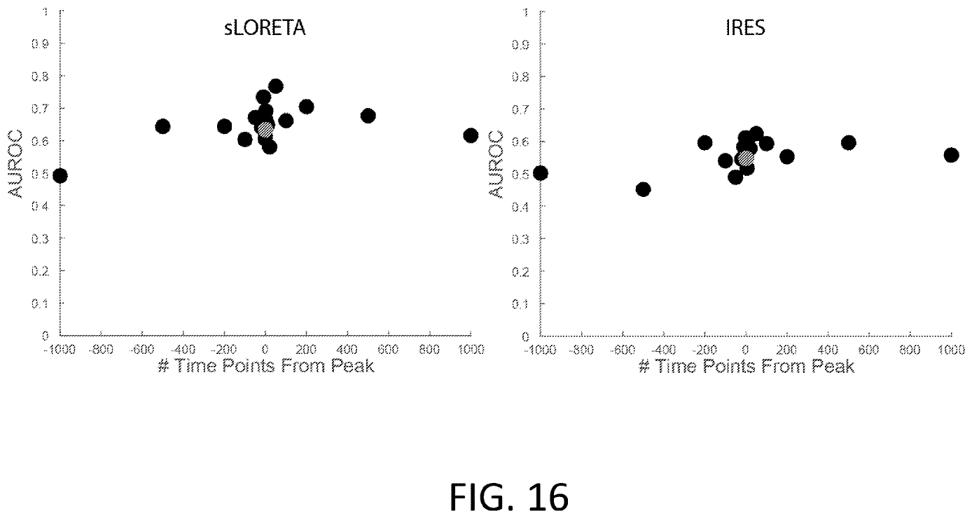

FIG. 17: Clinical decision support system workflow. TEDIE reconstructs the location of all the seizure onsets or spikes for a patient. The center of mass is computed for each seizure onset or spike, and the average center of mass of the seizures onsets or spikes is calculated. dCOM is computed and shown (*) compared to the historical data. The probability of seizure freedom is then calculated using K nearest neighbors where K, in this case, is 6.

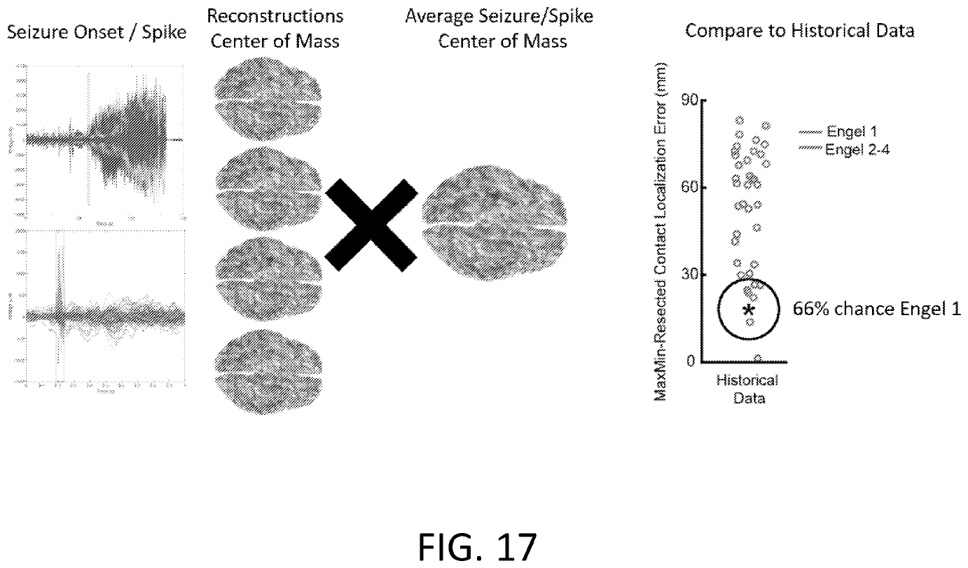

### DETAILED DESCRIPTION

Section headings as used in this section and the entire disclosure herein are merely for organizational purposes and are not intended to be limiting.

All publications, patent applications, patents and other references mentioned herein are incorporated by reference in their entirety.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Reduce” or other forms of the word, such as “reducing” or “reduction,” generally means a lowering of an event or characteristic. It is understood that this is typically in relation to some standard or expected value, in other words it is relative, but that it is not always necessary for the standard or relative value to be referred to.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician (e.g., physician).

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Certain methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Systems and Methods for Brain Source Localization

As described further herein, TEDIE is a propagating source reconstruction algorithm that accurately reconstructs the location and size of moving neural sources. TEDIE was validated by reconstructing a variety of synthetic neural sources with known locations and sizes, and TEDIE outperformed other previously developed algorithms in reconstructing synthetic neural sources. TEDIE was also validated using stereo-EEG recordings, and TEDIE's seizure onset zone reconstructions were closer to the electrode contact within the resected brain region for seizure-free patients compared to not seizure-free patients. Further, TEDIE generated more accurate, focal, and interpretable dynamic reconstructions (movies) of seizures compared to other previously developed algorithms, and this may improve clinical localization of the EZ. Additionally, results of the present disclosure demonstrated the prospective clinical utility of TEDIE in differentiating between seizure-free and not seizure-free patients using the consistency of onset zone localizations across seizures. Further, TEDIE was used to delineate potential new surgical targets in 13/23 patients who are currently not seizure-free. Additionally, it was found that the percentage of seizures with an ATL source can be used as a biomarker that can inform the use of MTL ablations vs ATL resections in treating temporal lobe epilepsies. Collectively, the results of the present disclosure demonstrate that TEDIE is an important new tool that can improve clinical EZ localization and potentially lead to more surgical candidates and better surgical outcomes from resection and ablation surgeries to treat epilepsy.

Manual localization of the EZ is a difficult and time-consuming process that is prone to bias. Manual localization leads to resection or ablation surgery only ˜55% of the time, and leads to seizure-freedom in patients who undergo surgery only ˜60% of the time. Nonetheless, patients who respond well to surgery can go from having debilitating seizures every day to seizure-freedom, which greatly improves quality of life and dramatically reduces the cost of treatment. Therefore, new tools that can reduce bias and improve localization of the EZ are expected to lead to more surgeries and improve surgical outcomes and thereby have a substantial clinical impact.

As described further herein, TEDIE can reduce bias and improve EZ localization by providing clinicians with a dynamic movie of seizure initiation and propagation mapped onto patient-specific brain geometry. Such visualization allows clinicians to investigate simultaneously many time points in an intuitive visualization of neural activity. TEDIE will thus reduce the bias in selecting which time points to investigate by reconstructing and displaying activity at all the time points. Further, TEDIE will reduce surgical planning bias, where clinicians currently qualitatively create surgical plans to remove only the brain tissues that surround the electrode contacts that record epileptogenic signals, while TEDIE provides clinicians quantitative maps of epileptogenic signals on patient-specific brain geometry to plan surgery.

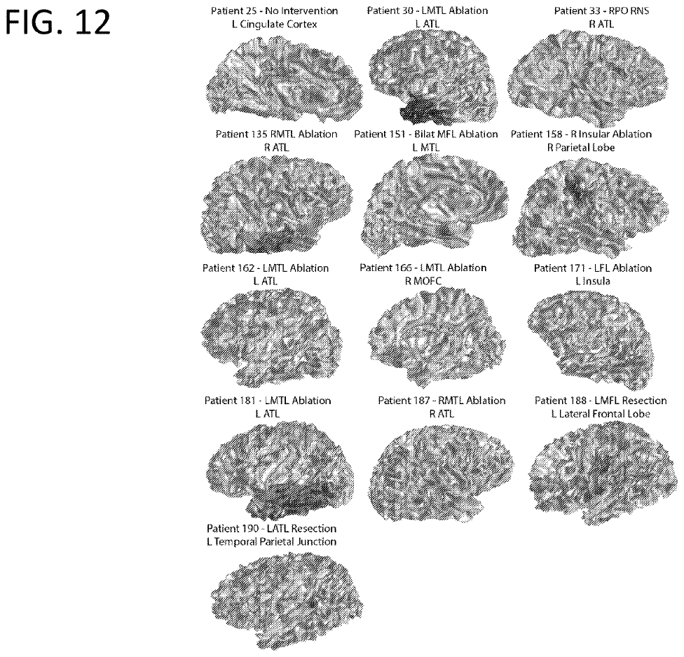

As described further herein, TEDIE can increase the proportion of patients who are deemed good surgical candidates and improve the outcomes of subsequent surgery. The consistency of seizure onset zones identified using TEDIE differentiated between seizure-free and not seizure-free patients in 810% of extratemporal lobe cases, and using the proportion of seizures with ATL sources differentiated 87% of MTL ablation cases. Both metrics can be calculated prospectively, and, if incorporated into clinical practice, may increase the seizure-freedom rates following extratemporal lobe (current seizure-freedom=50%) and temporal lobe epilepsy cases (current seizure-freedom=66%). Additionally, TEDIE identified new targets for therapy in 13/23 patients who are currently not seizure-free (FIG. 12). Thus, TEDIE can aid in developing new hypotheses that can be further investigated in especially difficult cases. Finally, in one patient who did not undergo surgery because a clear seizure onset zone could not be determined, TEDIE identified a consistent cingulate seizure onset zone, which explained the rapid bilateral perceived seizure onset observed during clinical evaluation. Therefore, TEDIE may also have clinical utility in identifying candidates for surgery who originally were determined to be poor surgical candidates. However, additional work is needed to quantify the proportion of additional patients that are good surgical candidates using TEDIE.

Many source localization algorithms have been developed over the past three decades to isolate the seizure onset zone. Early algorithms either localized a fixed number of dipole sources or determined the likelihood that dipoles at each location in the brain contributed to the recorded signals. These algorithms were subsequently implemented on stereo-EEG recordings with encouraging results (70% accuracy of differentiating between seizure free and not seizure free patients). However, recent developments in dynamic source reconstruction show that there is information encoded in the temporal dynamics of neural recordings that can improve source localization and estimate the size of the neural signal generators. TEDIE vastly outperformed static source localization algorithms in localizing known synthetic sources, localizing seizure onset zones, delineating accurate focal seizure onset zones, and generating interpretable movies of neural activity over time. Similar to previous dynamic localization algorithms, TEDIE outperformed static algorithms by a large margin. However, previous dynamic localization algorithm did not account for the propagation and growth of neural signal generators over time, while TEDIE did. Therefore, TEDIE is the only dynamic source reconstruction algorithm that can reconstruct the location and size of neural sources over time.

As described further herein, TEDIE can also be used as a clinical decision support system that determines the probability that a patient will be seizure free after resection or ablation surgery to treat epilepsy. Patients with medication-resistant epilepsy may undergo surgery to remove or ablate the brain tissue where seizures originate (i.e., the epileptogenic zone, EZ). In successful cases this procedure can be curative rendering the patient seizure free. However, it is often challenging to predict which patients will benefit from surgery, which of course comes with risks and costs. Thus, as described further herein, the present disclosure provides a novel system and method to calculate a probability that a patient will be seizure free after resection or ablation surgery.

In accordance with the above, embodiments of the present disclosure generally include a method for reconstructing propagating neural activity over time. In some embodiments, the method includes determining location and size estimates for a plurality of active neural sources based on neural activity at a plurality of time points. In some embodiments, neural activity is recorded using a plurality of electrodes (e.g., sEEG, EEG, or HD-EEG). In some embodiments, the method includes clustering adjacent populations of neural sources together to generate time-dependent clusters of neural sources. In some embodiments, the method includes determining a temporal average of the location and size of each cluster, In some embodiments, the method includes optimizing the location and size of each cluster by minimizing error between reconstructed and recorded signals. In some embodiments, the method includes generating a neural source likelihood map corresponding to likelihood that an active neural source is present at a specific location and at a specific time. As would be recognized by one of ordinary skill in the art based on the present disclosure, the method for reconstructing propagating neural activity over time can include one or more of the aforementioned steps in any order or arrangement.

In some embodiments, the method further includes repeating the step of determining a temporal average of the location and size of each cluster. In some embodiments, the method further includes repeating the step of optimizing the location and size of each cluster by minimizing error between reconstructed and recorded signals. In some embodiments, the method further includes repeating the step of determining a temporal average of the location and size of each cluster, and repeating the step of optimizing the location and size of each cluster by minimizing error between reconstructed and recorded signals.

In some embodiments, the method further includes minimizing error between a reconstructed and average location. In some embodiments, the method further includes minimizing error between a reconstructed and average size. In some embodiments, the method further includes minimizing error between a reconstructed and average location, and minimizing error between a reconstructed and average size.

In some embodiments, the method minimizes distance between neural sources in a cluster of neural sources across time. In some embodiments, the method minimizes changes in size between neural sources in a cluster of neural sources across time. In some embodiments, the method minimizes distance between neural sources in a cluster of neural sources across time, and minimizes changes in size between neural sources in a cluster of neural sources across time. In some embodiments, neural activity (e.g., neural activity at a plurality of time points) includes a patient-specific interictal spike and/or seizure recording(s). In some embodiments, the active neural source corresponds to a seizure onset zone. In some embodiments, neural activity is recorded using a plurality of electrodes (e.g., sEEG, EEG, or HD-EEG).

In some embodiments, the methods of the present disclosure include generating a neural source likelihood map corresponding to the likelihood that an active neural source is present at a specific location and at a specific time. In some embodiments, the method includes projecting the likelihood map onto a map of a patient's brain anatomy.

As described further herein, the methods and systems of the present disclosure have various clinical utility. For example, in some embodiments, the methods of the present disclosure can be used to diagnose a patient as being seizure-free or not seizure-free. In some embodiments, the methods of the present disclosure can be used to identify an active neural source as a target for surgical intervention. In some embodiments, neural activity at a plurality of timepoints is obtained by determining the location and size estimates for one or more neural sources. In some embodiments, determining location and size estimates comprises obtaining recordings from a plurality of electrodes that are compatible with electroencephalography (EEG), stereo-electroencephalography (sEEG), and/or magnetoencephalography (MEG).

As described further herein, the methods of the present disclosure demonstrated enhanced ability to reconstruct propagating and expanding neural sources over time compared to other currently available methods. For example, the methods of the present disclosure are capable of identifying an active neural source with a Jaccard index of at least about 0.5. In some embodiments, the methods of the present disclosure are capable of identifying an active neural source with a Jaccard index of at least about 0.6. In some embodiments, the methods of the present disclosure are capable of identifying an active neural source with a Jaccard index of at least about 0.7. In some embodiments, the methods of the present disclosure are capable of identifying an active neural source with a Jaccard index of at least about 0.8. In some embodiments, the methods of the present disclosure are capable of identifying an active neural source with a Jaccard index of at least about 0.9.

In some embodiments, the methods of the present disclosure are capable of identifying an active neural source with a mapped percentage of at least about 55%. In some embodiments, the methods of the present disclosure are capable of identifying an active neural source with a mapped percentage of at least about 60%. In some embodiments, the methods of the present disclosure are capable of identifying an active neural source with a mapped percentage of at least about 65%. In some embodiments, the methods of the present disclosure are capable of identifying an active neural source with a mapped percentage of at least about 70%. In some embodiments, the methods of the present disclosure are capable of identifying an active neural source with a mapped percentage of at least about 75%. In some embodiments, the methods of the present disclosure are capable of identifying an active neural source with a mapped percentage of at least about 80%. In some embodiments, the methods of the present disclosure are capable of identifying an active neural source with a mapped percentage of at least about 85%. In some embodiments, the methods of the present disclosure are capable of identifying an active neural source with a mapped percentage of at least about 90%. In some embodiments, the methods of the present disclosure are capable of identifying an active neural source with a mapped percentage of at least about 95%.

In some embodiments, the methods of the present disclosure are capable of identifying an active neural source with a center of mass localization error of no more than about 6.0 mm. In some embodiments, the methods of the present disclosure are capable of identifying an active neural source with a center of mass localization error of no more than about 5.5 mm. In some embodiments, the methods of the present disclosure are capable of identifying an active neural source with a center of mass localization error of no more than about 5.0 mm. In some embodiments, the methods of the present disclosure are capable of identifying an active neural source with a center of mass localization error of no more than about 4.5 mm. In some embodiments, the methods of the present disclosure are capable of identifying an active neural source with a center of mass localization error of no more than about 4.0 mm. In some embodiments, the methods of the present disclosure are capable of identifying an active neural source with a center of mass localization error of no more than about 3.5 mm. In some embodiments, the methods of the present disclosure are capable of identifying an active neural source with a center of mass localization error of no more than about 3.0 mm. In some embodiments, the methods of the present disclosure are capable of identifying an active neural source with a center of mass localization error of no more than about 2.5 mm. In some embodiments, the methods of the present disclosure are capable of identifying an active neural source with a center of mass localization error of no more than about 2.0 mm.

Embodiments of the present disclosure also include a method for determining probability of post-surgical seizure freedom in a patient. In accordance with these embodiments, the method includes performing epileptogenic zone localization in the patient using any of the methods described herein. In some embodiments, the method includes determining average epileptogenic zone location based on center of mass of each epileptogenic zone. In some embodiments, the method includes determining distance between the average epileptogenic zone location and the center of mass of each epileptogenic zone (dCOM value). In some embodiments, the method includes comparing the average epileptogenic zones locations from patients post-surgery to the patient to determine the probability of post-surgical seizure freedom in the patient. As would be recognized by one of ordinary skill in the art based on the present disclosure, the method for determining probability of post-surgical seizure freedom in a patient can include one or more of the aforementioned steps in any order or arrangement.

In some embodiments, the method further includes determining a confidence interval associated with the probability of post-surgical seizure freedom in the patient based on historical data of average epileptogenic zones locations in a plurality of post-surgical patients. In some embodiments, performing epileptogenic zone localization includes determining patient-specific interictal spike and/or seizure recordings. In some embodiments, the method further includes compiling a plurality of average epileptogenic zone locations from patients who are and are not seizure-free into a database. In some embodiments, the method further includes creating a classification system that outputs a score indicating whether the patient will or will not benefit from surgical intervention. In some embodiments, the classification system comprises assessing an Engel score and/or an ILAE score.

Embodiments of the present disclosure also include a method for determining probability of post-surgical seizure freedom in a patient undergoing mesial temporal ablation therapy. In accordance with these embodiments, the method includes performing epileptogenic zone localization in the patient using any of the methods described herein. In some embodiments, the method includes determining the proportion of the patient's epileptogenic zone location originating in the patient's neocortical anterior temporal lobe. In some embodiments, the method includes comparing the proportion of epileptogenic zone locations comprising neocortical anterior temporal lobe onset from patients post-surgery to the patient to determine probability of post-surgical seizure freedom in the patient. As would be recognized by one of ordinary skill in the art based on the present disclosure, the method for determining probability of post-surgical seizure freedom in a patient undergoing mesial temporal ablation therapy can include one or more of the aforementioned steps in any order or arrangement.

In some embodiments, the method further comprises determining a confidence interval associated with the probability of post-surgical seizure freedom in the patient based on historical data of average epileptogenic zones locations in a plurality of post-surgical patients. In some embodiments, the method further comprises compiling a plurality of average epileptogenic zone locations from patients who are and are not seizure-free into a database. In some embodiments, the method further comprises creating a classification system that outputs a score indicating whether the patient will or will not benefit from surgical intervention. In some embodiments, the classification system comprises assessing an Engel score and/or an ILAE score.

The systems and methods of the present disclosure can be implemented in hardware, software, firmware, or combinations of hardware, software and/or firmware. In some examples, the systems and methods described in this specification may be implemented using a non-transitory computer readable medium storing computer executable instructions that when executed by one or more processors of a computer cause the computer to perform operations. Computer readable media suitable for implementing the systems and methods described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system or method described in this specification may be located on a single device or computing platform or may be distributed across multiple devices or computing platforms.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

### 3. Materials and Methods

Data from 46 patients with drug resistant epilepsy were retrospectively analyzed from Duke Hospital (12 Patients) and the Hospital of the University of Pennsylvania (HUP) (34 patients). All patients provided written informed consented to data collection and sharing, and in this study, secondary research was conducted on deidentified data approved by the Duke University Institutional Review Board. All patients included in this study were implanted with stereo-EEG electrodes and had structural T1 MRI, post implantation CT, and stereo-EEG recording data available. A summary of the patient demographics is provided in Tables 1 and 2.

TABLE 1
Summary of patient demographics.

| Outcome | Engel 1 | Engel 2-4 |
|---|---|---|
| Number of Patients | 23 | 23 |
| Sex | | |
| Female | 12 | 13 |
| Male | 11 | 10 |
| Target | | |
| Temporal | 16 | 13 |
| Frontal | 4 | 8 |
| Parietal | 2 | 1 |
| Insular | 1 | 1 |
| Therapy | | |
| Resection | 10 | 5 |
| Ablation | 13 | 13 |
| No Intervention | 0 | 3 |
| Neuromodulation | 0 | 2 |

TABLE 2
Patient specific demographics.

| | | | | | # |
|---|---|---|---|---|---|
| Patient | Engel | | | | Reconstructed |
| ID | Score | Target | Therapy | Sex | Seizures |
| 2 | No Inter- | L Temporal | No Inter- | M | 4 |
| | vention | | vention | | |
| 3 | 1 | R MFL | Resection | F | 4 |
| 7 | 2 | L Frontal | Resection | F | 4 |
| 19 | 1 | L Parieto- | Resection | M | 2 |
| | | Occipital | | | |
| 20 | 1 | L MTL | Ablation | F | 3 |
| 24 | 1 | R MTL | Resection | F | 12 |
| 25 | No Inter- | L Temporo- | No Inter- | F | 8 |
| | vention | Parietal | vention | | |
| 26 | No Inter- | L Frontal | No Inter- | M | 6 |
| | vention | | vention | | |
| 27 | 1 | L MTL | Ablation | F | 5 |
| 30 | 2 | L MTL | Ablation | F | 5 |
| 31 | 3 | R Temporo- | ANT DBS | F | 3 |
| | | Parietal | | | |
| 33 | 3 | R Parieto- | RNS | F | 3 |
| | | Occipital | | | |
| 112 | 3 | R Frontal | Ablation | F | 5 |
| 116 | 1 | R MTL | Ablation | F | 3 |
| 117 | 1 | L Temporal | Resection | M | 3 |
| 130 | 1 | L MFL | Ablation | F | 5 |
| 132 | 3 | L Temporal | Ablation | F | 4 |
| 134 | 1 | R Frontal | Resection | M | 1 |
| 135 | 2 | R MTL | Ablation | M | 2 |
| 138 | 4 | L MTL | Ablation | M | 5 |
| 139 | 1 | L Parietal | Ablation | M | 3 |
| 140 | 1 | L MTL | Ablation | F | 3 |
| 141 | 1 | R MTL | Ablation | M | 5 |
| 144 | 1 | R Temporal | Resection | M | 5 |
| 146 | 1 | R Temporal | Resection | M | 3 |
| 148 | 1 | L Temporal | Ablation | M | 5 |
| 150 | 1 | R Insular | Ablation | M | 5 |
| 151 | 2 | R MFL | Ablation | M | 5 |
| 157 | 1 | L MTL | Ablation | M | 5 |
| 158 | 3 | R Insular | Ablation | M | 1 |
| 160 | 1 | R Temporal | Resection | F | 3 |
| 162 | 3 | L MTL | Ablation | F | 5 |
| 164 | 1 | L MTL | Ablation | F | 3 |
| 165 | 3 | R MTL | Ablation | F | 0 |
| 166 | 3 | L Temporal | Resection | M | 2 |
| 171 | 2 | L Frontal | Ablation | M | 4 |
| 172 | 2 | L Frontal | Ablation | F | 5 |
| 173 | 1 | R Temporal | Resection | F | 5 |
| 177 | 1 | R Temporal | Resection | F | 3 |
| 179 | 3 | L Frontal | Resection | F | 2 |
| 180 | 1 | L Frontal | Ablation | F | 5 |
| 181 | 3 | L Temporal | Ablation | F | 5 |
| 185 | 1 | L MTL | Ablation | M | 5 |
| 187 | 2 | R MTL | Ablation | M | 5 |
| 188 | 3 | L Frontal | Resection | F | 5 |
| 190 | 3 | L MTL | Resection | M | 3 |

Patient-specific head modeling. For each patient in the cohort, a patient-specific head model was generated using patient-specific neuroimaging and a semi-automated head modeling pipeline with four steps: 1) geometry creation, 2) defining tissue electrical properties, 3) electrode generation, and 4) finite element model generation. An overview of patient specific head modeling is provided below and additional details are provided in the material and methods section below.

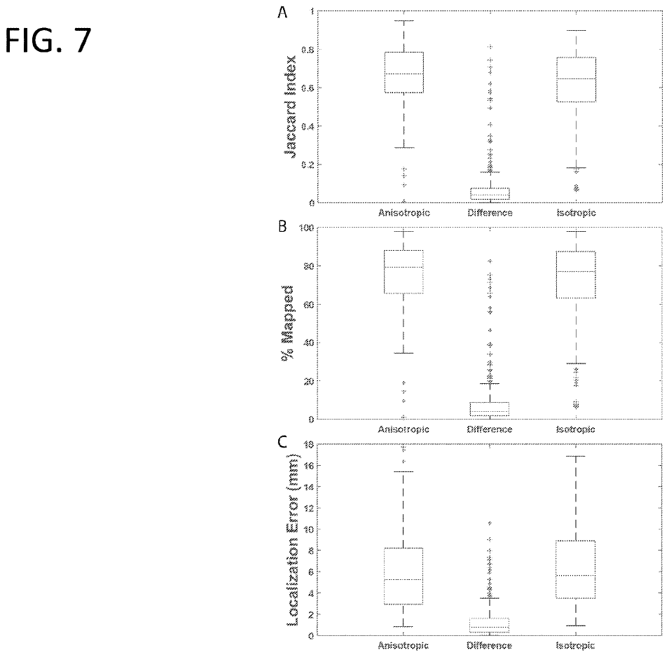

The model geometry was created by extracting skin, white matter, and amygdalohippocampal surfaces from the T1 MRI using FSL's (fsl.fnrib.ox.ac.uk) BET tool and freesurfer's (surfer.nmr.mgh.harvard.edu/) recon-all function. The brain surfaces were then smoothed by decimating and upsampling the mesh in meshlab. The electrical properties of the head models were defined by segmenting the T1 MRI using FSL's FAST and BET tools and assigning isotropic tissue conductivities based on tissue type. Isotropic models were created because diffusion weighted MRI was not available for all patients, and minimal differences in source localization were found between the anisotropic and isotropic models (FIG. 7).

The electrode geometries were generated using the post-implantation CT and a library of PMT and ADTech electrode mesh geometries generated from spec sheets. Electrode contacts were localized using DEETO for Duke Patients and the publicly available dataset for HUP patients. Contact locations were coregistered to T1 MRI space and a line was fit to each set of electrode contacts. The corresponding stereo-EEG electrode geometry from a library was then placed along the line. Additionally, the head geometry, electrical conductivities, and electrode geometries were combined into a finite element model in SCIRun v5.0 (SCI Institute, University of Utah, Salt Lake City, UT). Dipole source models are appropriate for representing small regions of cortex, so for each electrode contact, a point current source was placed within the electrode geometry and the voltages generated were computed by a unit current source at every neocortical and amygdalohippocampal location (cortical locations). The principle of reciprocity was used to calculate the voltages at each electrode contact that were generated by orthogonal dipole sources at each cortical location. The voltages were scaled by the area of each mesh element to maintain a uniform dipole moment density across the brain, and the voltages were compiled into two leadfield matrices: one where the dipoles were oriented orthogonal to the cortical surface (leadfield) and one that included three orthogonal constituent dipoles (vector leadfield).

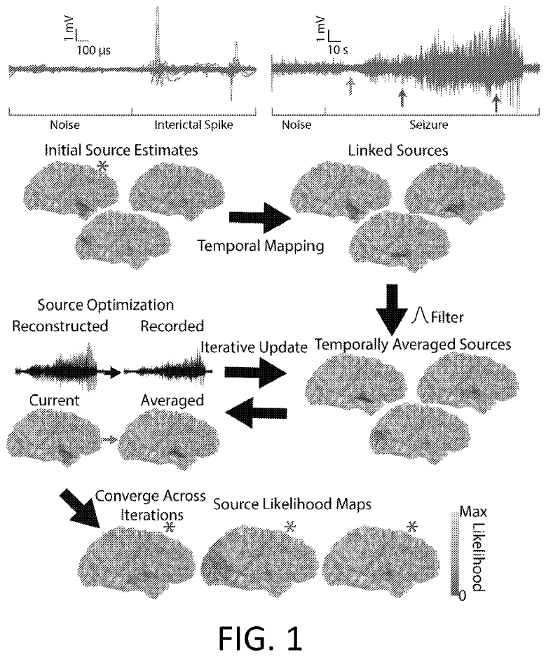

TEDIE (TEmporally Dependent Iterative Expansion). A novel approach was developed to optimize the location and size of moving neural sources called TEmporally Dependent Iterative Expansion (TEDIE). TEDIE uses the seizure recording, leadfield matrix, and cortical mesh geometry to reconstruct propagating neural sources. There are five steps to TEDIE: 1) initial source estimation, 2) source linking, 3) temporal source averaging, 4) source optimization, 5) source likelihood map generation. An overview of TEDIE is provided in FIG. 1.

TEDIE generates initial source estimates by determining the location and size of up to five neural sources that minimize the difference between the recorded and simulated voltages. For each time, TEDIE determines the location of a dipole source that best reproduces the recorded voltages. TEDIE then iteratively expands the size of the source from a single element of the cortical mesh by adding neighboring elements on the cortical mesh. TEDIE determines the source size and strength that minimize the difference between the recorded and simulated voltages at each time. If the mean difference between the recorded voltages and the voltages generated by the estimated source(s) is greater than a noise floor (2× mean voltage magnitude during 30 s before seizure), then TEDIE will place another source in the brain with size and location to accounts for the voltages not captured by the initial source. TEDIE repeats this process until the mean difference between the recorded voltages and the voltages generated by the simulated sources is below the noise floor or five sources are placed in the brain.

TEDIE then links the spatially close (<4 cm) initial source estimates across time. For each time TEDIE iterates through all source estimates and compares the location of the source's origin to the location of source clusters defined by previous times. If the distance between the source and a cluster is <4 cm, TEDIE adds the source to the cluster, but if the distance between the source and all clusters is >4 cm, TEDIE creates a new cluster with a location defined by the source's location. TEDIE then links sources that are in the same cluster and adjacent in time. TEDIE also constrain the source linking such that each source can only map to a single other source at another time.

For each source, TEDIE determines a temporally averaged source by applying a Gaussian filter (σ=2 time points) over time on the size and location of linked sources. This creates a source estimate that is dependent on other source estimates within a short window of time.

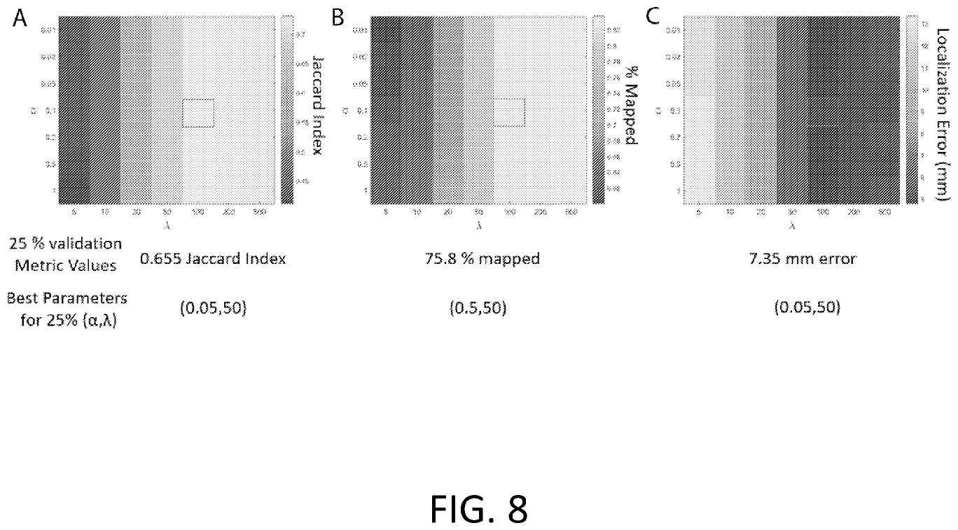

TEDIE uses the temporally averaged sources and sEEG recordings to optimize a new set of source locations and spatial extents that minimize a cost function defined by the differences between the reconstructed and recorded neural signals and the current and average source parameters (eq 1).

$$c o s t = \vert XYZ ( J ) - G ( XYZ ( J ) ) {\vert}_{2} + α \vert R ( J ) - G ( R ( J ) ) {\vert}_{2} + λ \vert KJ - Φ {\vert}_{2} \qquad ( 1 )$$

where J is the estimated neural source, XYZ is an operator that returns the Cartesian coordinates of the center of the source, G is the Gaussian-weighted average operator across time, R is an operator that returns the spatial extents of the source, K is the leadfield matrix, Φ is the recorded signal, a is a hyperparameter that controls the relative importance of minimizing the change in source size over time, and, is a hyperparameter that controls the relative importance of minimizing the difference between the reconstructed and recorded signals. A α=0.1 and λ=100 were chosen using cross validation on the cohort of 12 patients from Duke (FIG. 8).

Finally, TEDIE generates source likelihood maps at each time point using a sliding window approach. Within a short window of time (100-400 time points), TEDIE displays the percentage of time that each cortical source is active on a 3D visualization of the cortex. These source likelihood maps can be played as a movie to visualize the dynamic changes in the size and location of the reconstructed neural sources over time.

Synthetic source modeling. The performance of TEDIE was evaluated using reconstructions with known ground truth neural sources. Twenty-seven different temporally dependent synthetic source models were created with three different propagation speeds, three different growth rates, and three signal to noise ratios (SNR). To create the synthetic sources, a starting cortical location surrounded by the implanted electrodes was selected for each patient. For each source, the leadfield contributions of each active cortical element were summed and the summed contributions were multiplied by a 10 Hz sine wave to generate a synthetic recording. Each synthetic recording had a duration of 1 s with a time step of 1 ms. A leadfield matrix and cortical mesh were used with a higher mesh density to create synthetic sources and synthetic recordings to avoid the “inverse crimes” where the leadfield matrix used for source reconstruction and the synthetic sources are generated by the same model and thus can lead to an exact solution which biases the results. Finally, Gaussian white noise was added to the simulations to adjust the signal to noise ratio (SNR) (6 dB, 10 dB, and 20 dB).

Synthetic source analysis. TEDIE was run along with two other source localization algorithms, sLORETA and IRES, on the 27 synthetic source models for each patient. An open source implementation of sLORETA was used and the open source spatiotemporal version of IRES was adapted to run on single time points. A fixed dipole leadfield was used for TEDIE and a vector leadfield was used for sLORETA and IRES. Three performance metrics were computed: Jaccard index, percentage mapped, and center of mass localization error, to assess the performance of TEDIE, sLORETA, and IRES. Jaccard index is the number of active cortical elements present in both the reconstructed source and the ground truth synthetic source divided by the total number of active elements in either the reconstructed source or the ground truth synthetic source. Percentage mapped is the number of active cortical elements present in both the reconstructed source and the ground truth synthetic source divided by the number of active elements in the ground truth synthetic source. Center of mass localization error is the distance between the weighted average locations of the reconstructed source and the ground truth synthetic source. Each reconstruction was comprised of 1000 time points, so the median of each metric across time was computed for each patient. The performance of TEDIE, sLORETA, and IRES were compared using an ANOVA and post hoc t-tests where each patient constituted a separate data point.

Stereo-EEG signal processing. To evaluate the performance of TEDIE on real neural data, seizure times were isolated to include 30 seconds before the seizure onset until seizure offset. Channels with obvious muscle artifacts or 60 Hz noise were omitted. The recordings were referenced to a common average reference, and applied a 1 Hz high pass filter.

Seizure reconstruction analysis. TEDIE was run on each seizure recording and movies of the reconstructed neural activity during the seizure were rendered. The seizure onset zone was quantified by including the reconstructions from the first 100 time points after seizure onset that had neural signals whose magnitude was greater than three times the standard deviation of the recorded voltages across all channels. The performance of TEDIE was quantified by comparing the distance between the seizure onset zone and the tissue that was removed (based on the locations of electrode contacts within the resected brain region), which was the only data available to quantify the surgery. The best-resected distance was quantified as the distance between the cortical element with the highest source likelihood and the nearest electrode contact within the resected brain region. The maxmin-resected distance was quantified as the maximum distance between the reconstructed cortical elements and their corresponding nearest electrode contact within the resected brain region. These two distance metrics capture information about how close the reconstructions were to the resection and how much of the reconstructed seizure onset zone was removed by the resection. Sensitivity analyses was conducted to assess the influences of cutoff threshold (minimum value of source likelihood that should be included in the source estimate varied from >1% to >50% of the time active) and seizure onset time was chosen that varied between 1 to 500 time points.

TEDIE's performance was quantified for each patient using the median distance across all seizures. A set of binary classifiers were created to determine whether the median distance metrics could be used to delineate between patients with seizure-free (Engel 1) and not seizure-free (Engel 2-4) outcomes. Classifiers were trained using the best-resected distance and maxmin-resected distance using the entire cohort of patients, patients with temporal lobe epilepsies, and patients with extratemporal lobe epilepsies. For each dataset and classifier, performance metrics were quantified of the area under the receiver operator curve (AUROC) and for maximum accuracy. Bootstrapping was employed to resample the seizures for each patient 1000 times to estimate the mean and 95% confidence interval of the AUROCs and maximum accuracy. The distances for seizure-free vs not seizure-free patients and temporal lobe vs extratemporal lobe epilepsies were compared using a 2-way ANOVA and post hoc t-tests.

Comparison of source reconstruction algorithms. To assess the accuracy and interpretability of TEDIE compared to current source localization algorithms, all the seizures were reconstructed with TEDIE, sLORETA, and IRES. A vector leadfield was used for sLORETA and IRES and the fixed dipole leadfield was used for TEDIE. The seizure onset zone was identified with TEDIE using the same methods described herein. However, sLORETA and IRES are static algorithms, and the seizure onset zone identified with sLORETA and IRES were quantified using the time point with the highest amplitude across all channels within the seizure onset times used for TEDIE. The distance of the highest amplitude reconstruction location to the nearest electrode contact within the resected brain region was quantified and the distances for seizure-free vs. not seizure-free patients were compared. The spatial diffusivity of the seizure onset zone reconstructions generated by each algorithm were also quantified. The spatial diffusivity quantifies how concentrated the source is by determining the radius of a sphere centered at the highest amplitude point in the source that captures 50% of the source power. Lastly, the temporal consistency of the source reconstructions were quantified by calculating the Jaccard index of the reconstructed sources every 50 time points, and for TEDIE, a sliding window with a width of 200 time points was used. Kruskal-Wallis tests and post hoc Wilcoxon rank sum tests were used to compare the distance between the reconstructions and the resections, the spatial diffusivity, and temporal overlap for each algorithm.

Seizure consistency quantification. To determine whether TEDIE might contribute to presurgical decision making, experiments were conducted to determine whether a patient is seizure-free after surgery based on the consistency of their seizure onset zone. The center of mass of each seizure onset zone was quantified using cutoff thresholds ranging from 1-50% and the average seizure location was computed by taking the mean of the center of masses of all the seizures from a patient. For each patient, the variability in seizures was quantified as the mean distance between the center of mass of each seizure and the average seizure location. The distances for seizure-free vs not seizure-free patients and temporal lobe vs extratemporal lobe epilepsies were compared using a 2-way ANOVA and post hoc t tests.

The performance of a classifier based on seizure onset consistency was quantified using AUROC and maximum accuracy, and the changes in AUROC as a function of the number of seizures used to quantify consistency was also quantified. The changes in AUROC for classifiers trained with two to five seizures was quantified. For each number of seizures, all the patients who had greater than or equal to the number of seizures available were included to determine the likelihood of surgical candidacy. Bootstrapping was used to resample the seizures for each patient 1000 times to estimate the mean and 95% confidence interval of the AUROCs.

New Target Identification. To determine whether TEDIE might contribute to improvement in current clinical practice, experiments were conducted to determine if TEDIE could generate new hypotheses for surgical targeting. New surgical targets were identified as those present in >40% of the seizure onsets for a patient. For patients with temporal lobe epilepsies, the proportion of their observed seizures that included anterior temporal lobe sources for a variety of cutoff thresholds (25%, 50%, 75%, and 99%) and seizure onset window sizes (2, 4, 6, 8, and 10 s) were also quantified. A Kruskal-Wallis test and post hoc one tailed Wilcoxon rank sum tests were used to compare the proportions of seizures with anterior temporal lobe sources for patients with Engel 1 outcomes after MTL ablations, patients with Engel 2-4 outcomes after MTL ablations, and patients with Engel 1 outcomes after ATL resections.

### 4. Examples

Successful surgery for patients with epilepsy require proper identification of seizure origin. Clinical identification of the seizure origin is accomplished using intracranial stereo-EEG, whereby ˜5-30 multi-contact electrodes are implanted, through small drill holes, into widespread regions of the brain. Despite technological advances in epilepsy monitoring and surgery, seizure freedom rates following surgery have not improved over the past decade, ˜60%. In part, seizure freedom rates have not improved because only qualitative analysis tools are used clinically.

Embodiments of the present disclosure include a novel source reconstruction method, termed “TEmporally Dependent Iterative Expansion” (TEDIE), that accurately reconstructs propagating and expanding neural sources over time. TEDIE iteratively optimizes the number, location, and size of neural sources to minimize the differences between the reconstructed and recorded stereo-EEG signals using temporal information to refine the reconstructions. The output of TEDIE is a movie of seizure activity projected onto patient-specific brain anatomy. Data from 46 epilepsy patients implanted with stereo-EEG electrodes were retrospectively analyzed at Duke Hospital (12 patients) and Hospital at the University of Pennsylvania (34 patients). TEDIE was validated by accurately reconstructing a variety of propagating and growing synthetic neural sources with known locations and sizes. It was found that TEDIE outperformed other algorithms (sLORETA and IRES) in reconstructing synthetic neural sources. TEDIE was also validated using seizure recordings and these data demonstrated that TEDIE's seizure onset zone reconstructions were closer to the electrode contacts within the resected brain region for seizure-free patients compared to not seizure-free patients. Further, TEDIE generated more accurate, focal, and interpretable dynamic reconstructions (movies can be made available upon request) of seizures compared to sLORETA and IRES. The results of the present disclosure also demonstrated the potential prospective clinical utility of TEDIE in differentiating between seizure-free and not seizure-free patients using the consistency of seizure onset zone reconstructions across seizures. Additionally, TEDIE was used to delineate potential new surgical targets in 13/22 patients who are currently not seizure-free. Furthermore, results demonstrated that the percentage of seizures with an anterior temporal lobe source reconstructed by TEDIE can be used as a biomarker to inform the use of mesial temporal lobe ablations or anterior temporal lobe resections in treating temporal lobe epilepsies. The results of the present disclosure demonstrate that TEDIE is a useful clinical tool that can greatly improve epileptogenic zone localization and improve outcomes following surgery.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

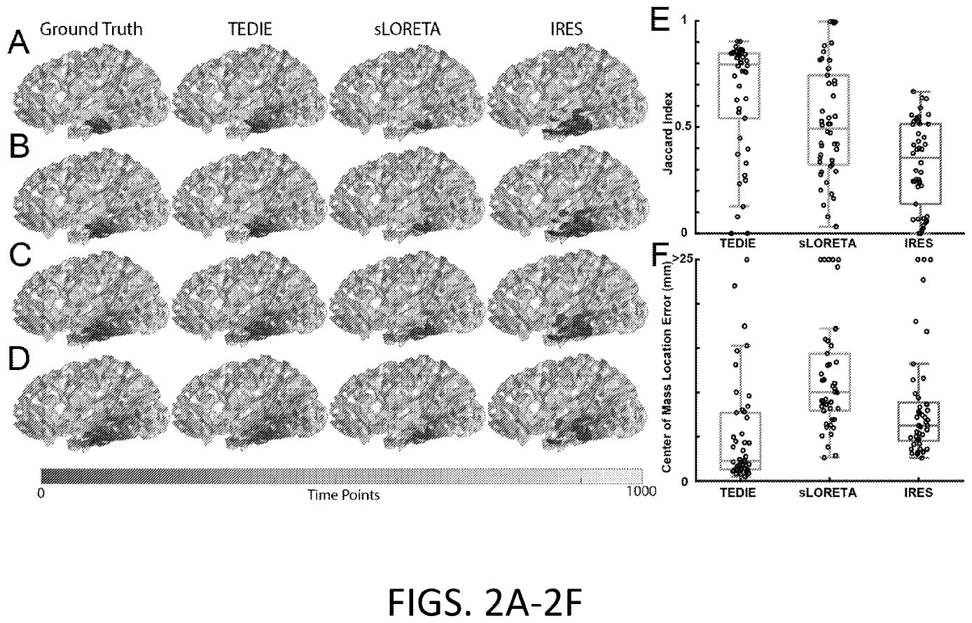

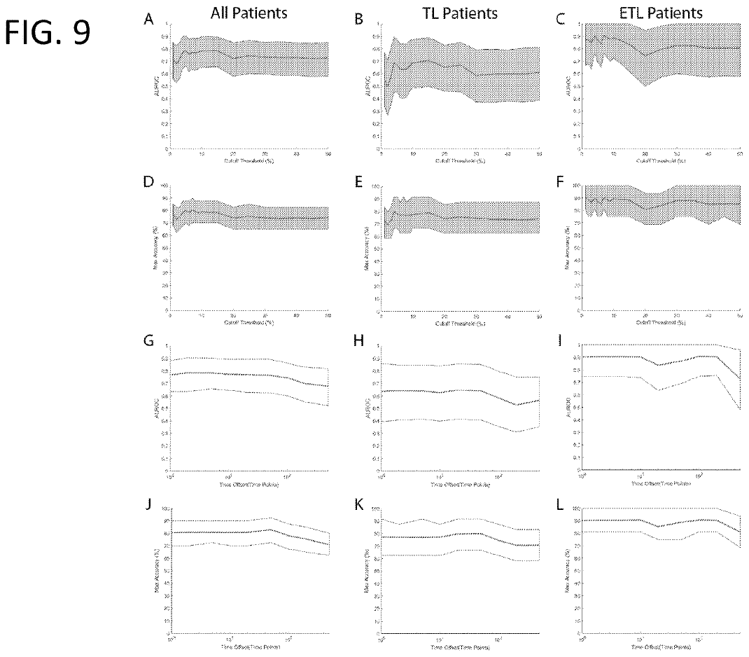

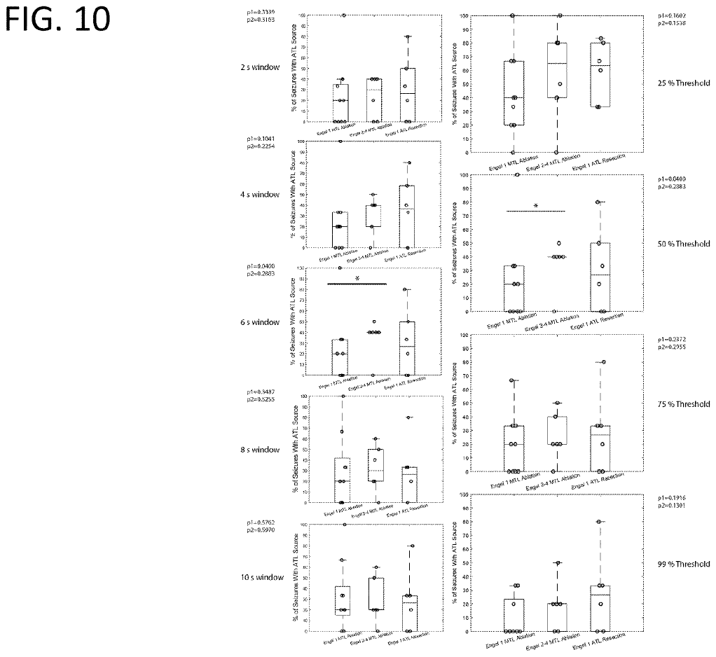

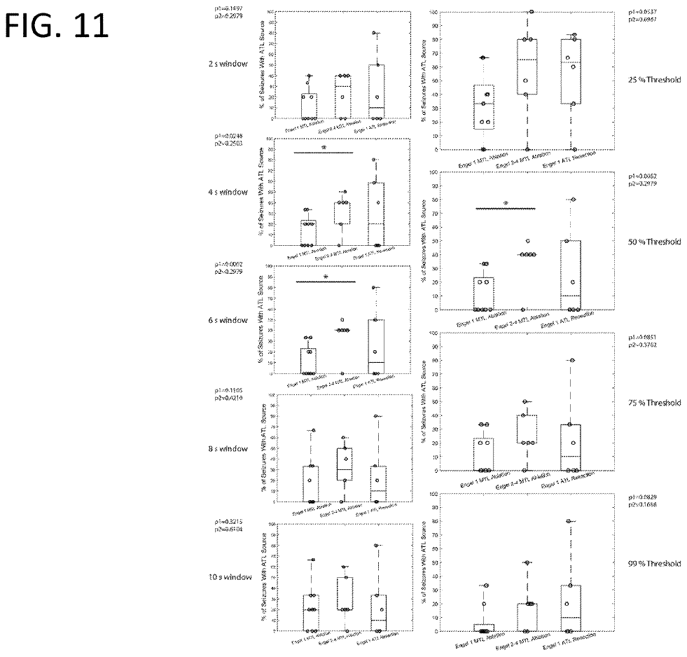

TEDIE outperforms other source localization algorithms in reconstructing synthetic neural sources. To validate TEDIE, reconstructions were compared to known ground truth synthetic neural sources where the locations and sizes of the sources over time were controlled. The voltages at each stereo-EEG electrode contact were stimulated for 46 patients generated by 27 different source types with different propagation speeds, growth rates, and SNRs and the performance of TEDIE was compared to two other source localization algorithms, sLORETA and IRES. TEDIE's reconstructions matched the ground truth sources better than sLORETA and IRES across the wide variety of different source types (FIGS. 2A-D; corresponding movies can be made available upon request). There was quantitatively greater overlap between the ground truth source and TEDIE reconstructions compared to the reconstructions from sLORETA and IRES (FIG. 2E, FIG. 9, and FIG. 10) as reflected in the larger Jaccard index (mean TEDIE=0.8, sLORETA=0.49, IRES=0.36, F(2,45)=21; p=2e-8) and percentage mapped (mean TEDIE=93%, sLORETA=50%, IRES=41%, F(2,45)=18; p=1e-7). Additionally, TEDIE reconstructions had a smaller center of mass localization error (mean TEDIE=2.3 mm, sLORETA=10 mm, IRES=6.3 mm, F(2,45)=8; p=6e-4) than sLORETA and IRES (FIG. 2F and FIG. 11).

### Example 2

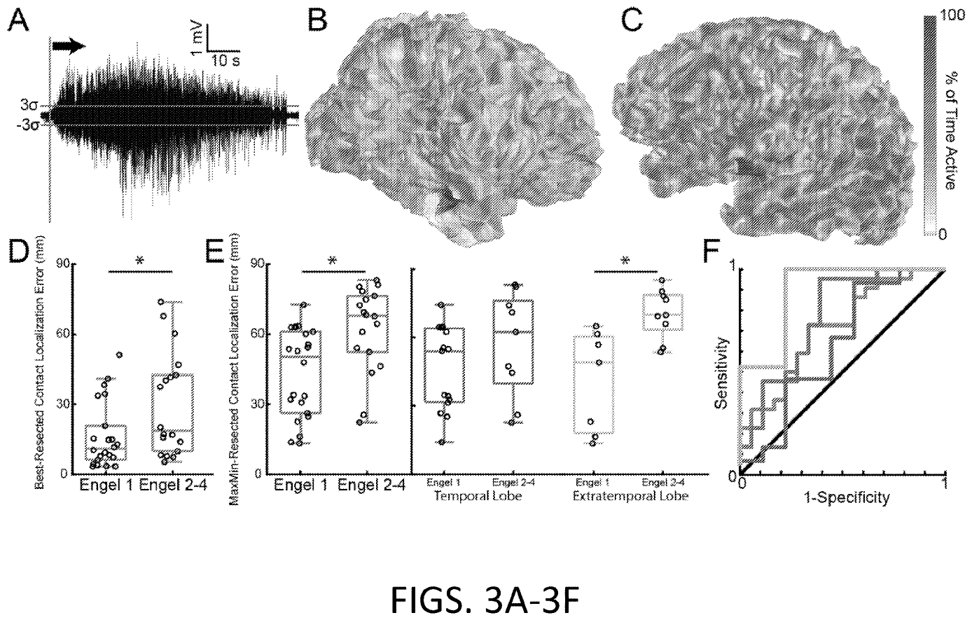

TEDIE reconstructions are closer to resections for Engel 1 patients compared to Engel 2-4 patients. After validating TEDIE using known synthetic sources, TEDIE was used to reconstruct neural activity during seizures. The neural activity was visualized during a seizure as a movie (available upon request). However, clinically, the entire propagation path of a seizure does not typically inform the EZ localization. Instead, only seizure onset is used in EZ localization and surgical planning. Therefore, the seizure onset zone was quantified using the TEDIE reconstructions in 40 patients with seizure recordings and surgical intervention data (FIG. 3A). The highest amplitude locations in the reconstructions were closer to the electrode contacts within the surgically removed regions of cortex for seizure-free compared to not seizure-free patients (FIGS. 3B-3D) (median distances=11.0 mm vs 18.6 mm; F(1,1,39)=4.69; p=0.04). Additionally, seizure-free patients had a smaller maximum distance of any reconstructed cortical source to the nearest electrode contact within the resected brain region compared to not seizure-free patients (FIG. 3E) (median distances=50.3 mm vs 67.9 mm; F(1,1,35)=10.55; p=0.002). When separately investigating temporal lobe and extratemporal lobe cases, there were large differences in reconstruction to resection distances for seizure-free and not seizure-free patients in extratemporal lobe cases (median distances=47.9 mm vs 68.2 mm; t(14)=3.5; p=0.003) but not for temporal lobe cases (median distances=52.7 mm vs 60.9 mm; t(22)=1.3; p=0.2) (FIG. 3E).

To quantify further the ability of TEDIE to discriminate between seizure-free and not seizure-free patients, a linear classifier was trained (FIG. 3F). The classifier built for extratemporal lobe cases performed exceedingly well (AUROC=0.90, Accuracy=88%), the classifiers built for all the patients performed adequately (AUROC_{Best}=0.71 and AUROCₘₐₓₘᵢₙ=0.77, Accuracy_{Best}=70% and Accuracyₘₐₓₘᵢₙ=80%), and the classifier built for temporal lobe cases performed modestly (AUROC=0.63, Accuracy=75%). A sensitivity analysis of classifier performance on cutoff threshold and seizure onset time was conducted, and a cutoff threshold between 5-15% led to the best classifiers with AUROCs>0.75 (FIGS. 9A-9F), while the performance of the classifier was stable (AUROC>0.75) until 200 time points (0.2 s-0.97 s) beyond the initially chosen seizure onset time (FIGS. 9G-9L).

### Example 3

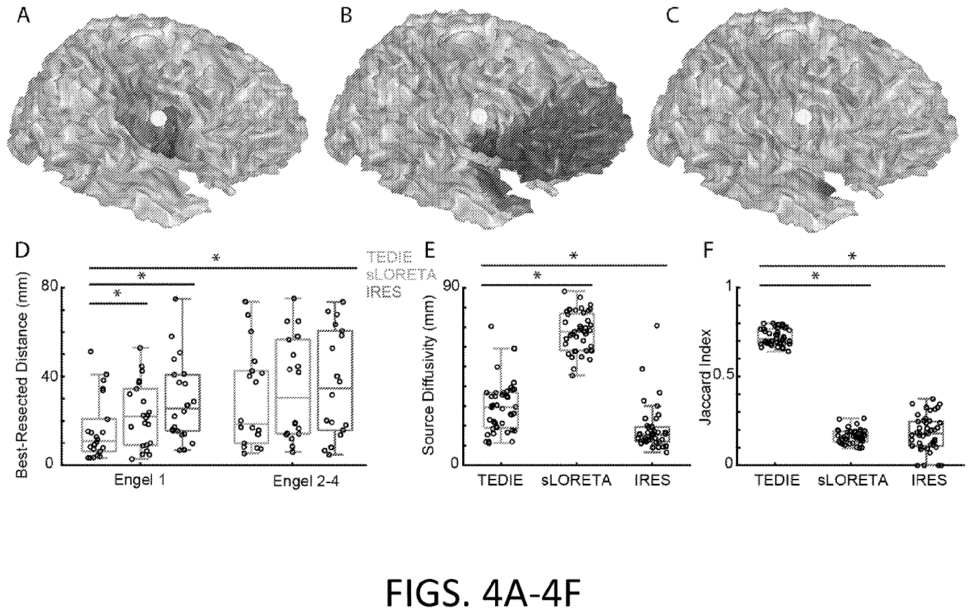

TEDIE reconstructions are more accurate and interpretable than sLORETA and IRES. Seizure reconstructions by TEDIE, sLORETA, and IRES were used to determine if there is additional clinical value in TEDIE compared to other algorithms. TEDIE generated more focal reconstructions of the seizure onset zone that were centered about the electrode contacts within the resected brain region compared to sLORETA and IRES for seizure-free patients (FIGS. 4A-4C). The highest amplitude points in the TEDIE reconstructions were closer to the electrode contacts within the resected brain region compared to the highest amplitude points in the sLORETA and IRES reconstructions (TEDIE=11 mm, sLORETA=21.9 mm, IRES=25.6 mm; χ²(2,39)=6.61; p=0.04) (FIG. 4D). The spatial diffusivity of each seizure onset zone reconstructed by TEDIE, sLORETA, and IRES were quantified. TEDIE had more focal onset zones compared to sLORETA but less focal onset zones compared to IRES (TEDIE=29.3 mm, sLORETA=67.6 mm, IRES=15.4 mm; χ²(2,162)=84.5; p=4e-19) (FIG. 4E). Additionally, the temporal consistency of the onset zones for each algorithm were quantified by comparing the reconstructions between time points. The temporal consistency can be interpreted as the interpretability of a movie of the seizure across time. TEDIE produced a substantially larger temporal consistency than sLORETA and IRES (TEDIE=0.71, sLORETA=0.16, IRES=0.18; χ²(2,179)=89.8; p=3e-20) (FIG. 4F; corresponding movie can be made available upon request).

### Example 4

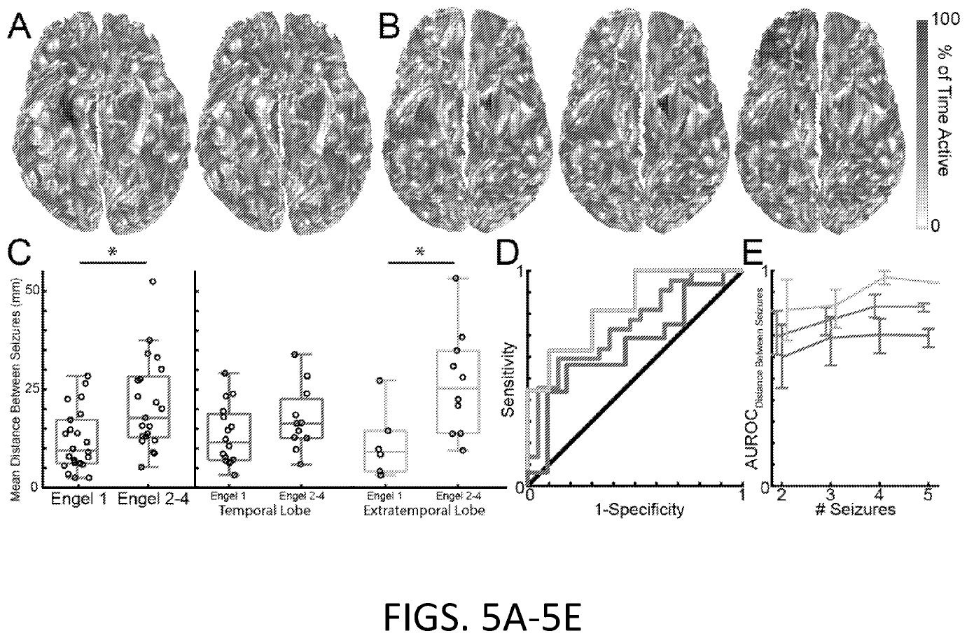

Consistency of reconstructed seizures can accurately predict postsurgical outcomes. While it is important to validate the performance of TEDIE on retrospective cases with surgical data, a useful source reconstruction tool would benefit from a biomarker to inform postsurgical outcomes without surgical data. Experiments were conducted to investigate the consistency of seizure localizations as a potential biomarker to predict a patient's surgical candidacy agnostic to the surgical data. Seizure-free patients had more consistent seizure onset zone localizations compared to not seizure-free patients (FIGS. 5A-5C) (median distance=9.36 mm vs 17.68 mm; F(1,1,42)=7.3; p=0.01). Additionally, there was a large difference in consistency of seizure onset zone localizations for seizure-free and not seizure-free patients with extratemporal lobe epilepsy (median distance=8.28 mm vs 24.43 mm; t(14)=2.5; p=0.03) but not for temporal lobe epilepsy (median distance=10.62 mm vs 15.49 mm; t(25)=1.4; p=0.2). A linear classifier was trained on the seizure onset zone consistency data, and the classifiers performed similarly well compared to the classifiers built using the distances from the resections (FIG. 5D). The classifier for extratemporal lobe cases performed exceedingly well (AUROC=0.85, Accuracy=81%), the classifier built for all the patients performed adequately (AUROC=0.75, Accuracy=72%), and the classifier built for temporal lobe cases performed modestly (AUROC=0.65, Accuracy=67%). Additionally, the performance of the classifiers based on the number of seizures used to train the classifier was investigated, and the classifiers for all patients and extratemporal lobe cases generally performed better with increasing numbers of seizures up to four seizures (FIG. 5E and Tables 4-6).

### Example 5

TEDIE identifies new surgical targets. TEDIE accurately reconstructed seizures and outperformed other source localization algorithms. However, to assess the potential clinical value of TEDIE to current clinical practice experiments were conducted to investigate whether TEDIE could identify new surgical targets for therapy in patients who were not currently seizure-free. The seizure onset zones were identified for the 23 patients who are not seizure-free. If more than 40% of the TEDIE reconstructed seizure onset zones for a patient showed the same source that was not surgically removed, the patients were classified as having an alternative surgical target. Across the 23 patients, new potential surgical targets were identified in 13 patients (Table 3 and FIG. 12).

TABLE 3
Novel Surgical Targets Predicted by TEDIE in Engel 2-4 Patients.

| Patient | | TEDIE Identified Seizure |
|---|---|---|
| ID | Previous Therapy | Onset Source |
| 25 | No Intervention | L Cingulate Cortex |
| 30 | L Mesial Temporal Lobe Ablation | L Anterior Temporal Lobe |
| 33 | R Temporal-Occipital RNS | R Anterior Temporal Lobe |
| 135 | R Mesial Temporal Lobe Ablation | R Anterior Temporal Lobe |
| 151 | R Mesial Frontal Lobe Ablation | L Mesial Temporal Lobe |
| 158 | R Insula | R Mesial Parietal Lobe |
| 162 | L Mesial Temporal Lobe Ablation | L Anterior Temporal Lobe |
| 166 | L Anterior Temporal Lobe | R Medial Orbitofrontal |
| | Resection | Cortex |
| 171 | L Frontal Lobe Ablation | L Insula |
| 181 | L Mesial Temporal Lobe Ablation | L Anterior Temporal Lobe |
| 187 | R Mesial Temporal Lobe Ablation | R Anterior Temporal Lobe |
| 188 | L Frontal Lobe Resection | L Lateral Frontal Lobe |
| 190 | L Mesial Temporal Lobe Ablation | L Parieto-Temporal |
| | | Junction |

TABLE 4
Necessary Number of Seizures All Data. One-sided empirical
p-values are calculated using the proportion of bootstrapped
AUROCS that are larger when defining consistency with more
seizures compared to fewer seizures, i.e., pval_{2vs3 seizures} =
sum(AUROC_{3 seizures} − AUROC_{2 seizures})/N (N is number of
times the distributions are resampled, in this case 1000).

| | 2 Seizure | 3 Seizures | 4 Seizures | 5 Seizures |
|---|---|---|---|---|
| 2 Seizure | X | 0.194 | 0.018 | 0.012 |
| 3 Seizures | X | X | 0.082 | 0.044 |
| 4 Seizures | X | X | X | 0.557 |
| 5 Seizures | X | X | X | X |

TABLE 5
Necessary Number of Seizures Temporal Lobe. One-sided empirical
p-values are calculated using the proportion of bootstrapped
AUROCS that are larger when defining consistency with more
seizures compared to fewer seizures, i.e., pval_{2vs3 seizures} =
sum (AUROC_{3 seizures} − AUROC_{2 seizures})/N (N is number of
times the distributions are resampled, in this case 1000).

| | 2 Seizure | 3 Seizures | 4 Seizures | 5 Seizures |
|---|---|---|---|---|
| 2 Seizure | X | 0.223 | 0.185 | 0.187 |
| 3 Seizures | X | X | 0.425 | 0.510 |
| 4 Seizures | X | X | X | 0.576 |
| 5 Seizures | X | X | X | X |

TABLE 6
Necessary Number of Seizures Extratemporal Lobe. One-sided
empirical p-values are calculated using the proportion of
bootstrapped AUROCS that are larger when defining consistency
with more seizures compared to fewer seizures, i.e., pval_{2vs3 seizures} =
sum(AUROC_{3 seizures} − AUROC_{2 seizures})/N (N is number of
times the distributions are resampled, in this case 1000).

| | 2 Seizure | 3 Seizures | 4 Seizures | 5 Seizures |
|---|---|---|---|---|
| 2 Seizure | X | 0.469 | 0.078 | 0.110 |
| 3 Seizures | X | X | 0 | 0 |
| 4 Seizures | X | X | X | 0.888 |
| 5 Seizures | X | X | X | X |

### Example 6

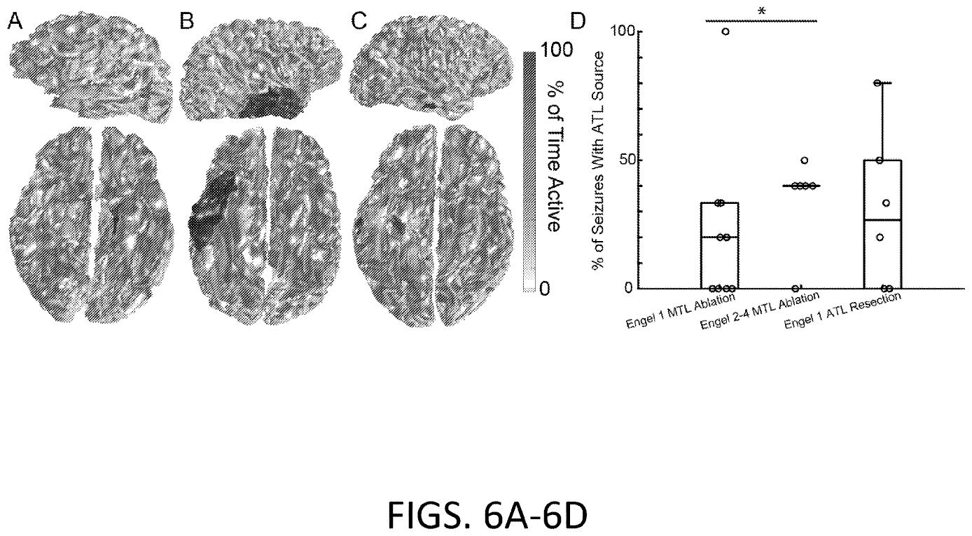

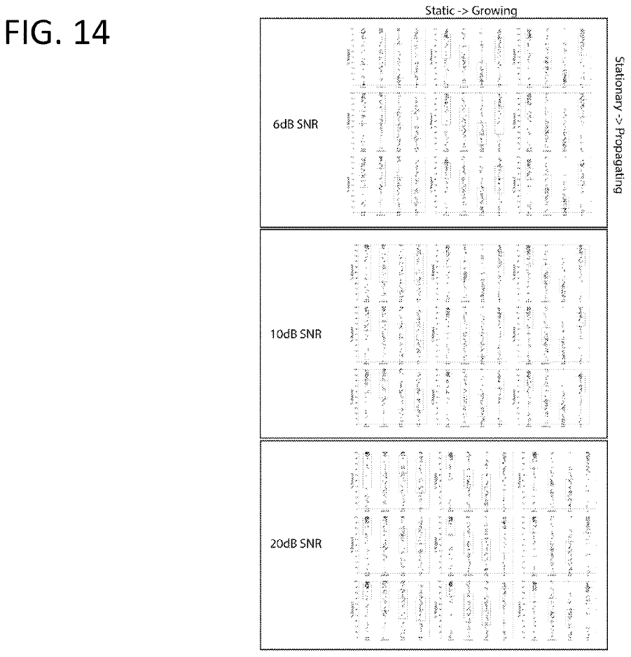

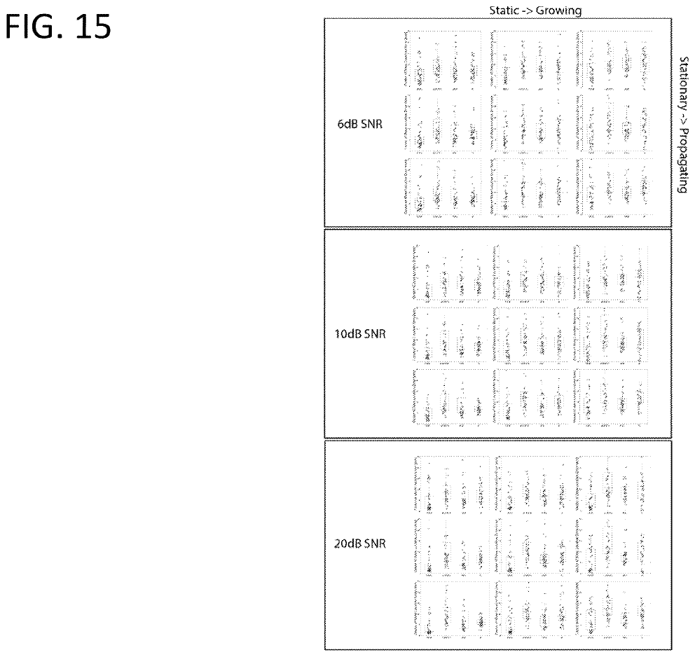

TEDIE can inform the use of anterior temporal lobe resections vs. mesial temporal lobe ablations. Many of the new surgical targets identified by TEDIE were anterior temporal lobe (ATL) sources (5/13) in patients who originally were treated with mesial temporal lobe (MTL) ablations. Additionally, both MTL ablations and ATL resections are used to treat suspected hippocampal onset seizures. Patient are known to have higher seizure-freedom rates with ATL resections compared to MTL ablations, but there are also higher complication rates with ATL resections compared to MTL ablations. Therefore, a biomarker that can determine whether a patient will respond to MTL ablation may have substantial clinical impact. The distance between the electrode contacts within the resected brain region and the TEDIE reconstructions for patients with temporal lobe epilepsy was not a good biomarker to distinguish between seizure-free and not seizure-free patients (FIG. 3E). However, the analysis of new surgical targets (Table 3), suggested that the percentage of seizures with lateral ATL sources could potentially distinguish between seizure-free and not seizure-free patients. The proportion of seizures with ATL sources for seizure-free patients from MTL ablations (not seizure-free patients from MTL ablations) were compared with seizure-free patients from ATL resections. Seizure-free patients had fewer ATL sources compared to not seizure-free patients (FIGS. 6A, 6B, and 6D, FIG. 14 and FIG. 15) (median % seizures with ATL sources=20% vs 40%; ranksum(13)=57; p=0.04). However, a difference between seizure-free patients with MTL ablations and ATL resections was not detected (median % seizures with ATL sources=20% vs 27%; ranksum(13)=67; p=0.3; power=0.09) because the variance in the proportion of seizures with ATL sources for the cohort of patients who received ATL resections was quite large (FIGS. 6C and 6D). Therefore, TEDIE can identify candidates for MTL ablation therapies using the proportion of seizures with ATL sources, and ATL resections appear to produce equivalent results across patients with or without ATL onset sources.

### Example 7

Clinical Decision Support System to Estimate Seizure Freedom Post Surgery. Surgery to remove or ablate the EZ requires first determining the location and spatial extent of the brain tissue where seizures originate. As described above, the function of TEDIE was validated by reconstructing the seizures and spikes (another form of epileptic neural activity) in 43 patients treated at Duke and University of Pennsylvania hospitals. It was found that patients with good surgical outcomes (Engel 1/2—seizure free or infrequent seizures) had localizations of the EZ using multiple seizures and/spikes that were tightly clustered in space (i.e., concordant). However, patients with poor surgical outcomes (Engel 3/4—minor improvements to no improvement in seizure frequency) had localizations of the EZ using multiple seizures and/spikes that were dispersed in space (i.e., discordant). Using this patient cohort, a logistic classifier was trained to predict whether patients had good vs poor surgical outcomes based on the consistency of EZ localizations from spikes and/or seizures. This simple classifier had an accuracy of 72% across all patients and 81% when considering only patients with extratemporal lobe epilepsies (FIG. 5). These results demonstrate the use of a source localization (e.g., with TEDIE) clinical decision support system to predict prospectively surgical outcomes.

For each patient, source localization (e.g., with TEDIE) localizes the EZ individually from each interictal spike and seizure recorded from a particular patient (as determined by a clinician and/or an automated detection algorithm), i.e., a patient-specific set of multiple EZ localizations. The center of masses of the EZs determined from each spike and/or seizure were then combined to determine the average localization per patient. The distance between the center of mass of the EZ localization from each individual spike and/or seizure were determined and the average EZ localization (dCOM) was also determined. The dCOM value is used by the clinical support system to calculate the probability of seizure freedom.

The probability of seizure freedom is calculated by finding the patients who had dCOM values closest to that of the patient under evaluation (i.e., K nearest neighbors) and using the outcomes of those patients (the percent of those patients who were seizure-free after surgery) to determine the probability that the patient under study will be seizure-free. The clinical support system is trained on historical data from all patients who had EZ localization using source localization (e.g., with TEDIE). Therefore, as source localization (e.g., with TEDIE) is used to analyze more epilepsy patients, these data are added to the algorithm, and the probability estimates are updated and the predictions will improve. Importantly, the number of patients who are seizure-free vs those who are not seizure-free will likely not be equal in the dataset, and thus the relative contributions of historical seizure-free patients and not seizure-free patients were scaled to account for the imbalance in sample sizes when calculating the proportion of patients who are seizure-free. Finally, the clinical support system calculates the confidence interval in the probability of seizure freedom using the multiple localized EZs from individual spikes and/or seizures. For each spike/seizure, a dCOM value was calculated and a probability of seizure freedom was determined. The multiple probabilities of seizure freedom were then used to calculate a mean and confidence interval for the probability of seizure freedom in the clinical support system.

Further examples of the clinical support system can include the use of other methods for calculating the probability of seizure freedom. For example, one the clinical support system can use a fixed interval of historical dCOM values to calculate the probability of seizure freedom (dCOM±interval). Another the clinical support system fits probability density functions to the dCOM values for patients who are seizure-free and those who are not seizure-free. A Bayesian method is then applied to determine the probability of seizure freedom given a new value of dCOM. In addition to different methods for calculating the probability of seizure freedom, further the clinical support systems of the present disclosure can use different outcome metrics beyond seizure-free and not seizure-free. For example, Engel Scores (1-4) or ILAE scores (1-6) can be used in the clinical support system and the probability of a patient achieving each of the different scores can be determined by the clinical support system.

In a further example, the clinical support system incorporates the historical data of resected electrode contacts to determine the probability of seizure freedom given a specific resection plan. When analyzing the 40 patients in the patient cohort with seizure data and the results of surgical intervention, it was found that patients who are seizure-free after surgery had seizures that were closer to the resected electrode contacts compared to those who are not seizure-free. A simple classifier had an accuracy of 80% across all patients and 88% when only considering patients with extratemporal lobe epilepsies (FIG. 3). In this clinical support system, a clinician will define either a volume of tissue or set of electrode contacts that are to be removed. The clinical support system calculates the distance between the EZ reconstructed by source localization (e.g., with TEDIE) and the volume of tissue or electrode contacts that will be removed (dEZR). Using the historical data of all patients analyzed with source localization (e.g., with TEDIE), the probability of seizure freedom is calculated by finding the patients who had dEZR values closest to that of the patient under evaluation. All subsequent analysis steps follow as in the original clinical support system. Further examples of this application of a clinical support system include various ways of calculating the distance between the EZ and the volume of tissue or electrode contacts that will be resected. The distance between the maximum amplitude point in the EZ and the planned resection, the distance between the center of mass of the EZ and the planned resection, the minimum distance between the EZ and the planned resection, the mean distance between the EZ and the planned resection, and the maximum distance between the EZ and the planned resection, or some combination of these metrics can be used in this clinical support system.

An additional example of the clinical support system of the present disclosure is as a classifier (Logistic classifier, Decision Tree, Support Vector Machine, Multilayer perceptron, and the like) where instead of yielding a probability of seizure freedom, the clinical support system outputs, for a given dCOM value, a binary good surgical candidate or poor surgical candidate outcome.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art.

1. A method for reconstructing propagating neural activity over time, the method comprising:
determining location and size estimates for a plurality of active neural sources based on neural activity at a plurality of time points;
clustering adjacent populations of neural sources together to generate time-dependent clusters of neural sources;
determining a temporal average of the location and size of each cluster;
optimizing the location and size of each cluster by minimizing error between reconstructed and recorded signals; and
generating a neural source likelihood map corresponding to likelihood that an active neural source is present at a specific location and at a specific time.

2. The method of claim 1, further comprising repeating the step of determining a temporal average of the location and size of each cluster, and/or repeating the step of optimizing the location and size of each cluster by minimizing error between reconstructed and recorded signals.

3. The method of claim 1, further comprising minimizing error between a reconstructed and average location, and/or minimizing error between a reconstructed and average size.

4. The method of claim 1, wherein the method minimizes distance between neural sources in a cluster of neural sources across time, and/or minimizes changes in size between neural sources in a cluster of neural sources across time.

5. (canceled)

6. The method of claim 1, wherein the neural activity comprises patient-specific interictal spike and/or seizure recordings.

7. The method of claim 1, further comprising projecting the likelihood map onto a map of a patient's brain anatomy.

8. The method of claim 1, wherein the active neural source corresponds to a seizure onset zone.

9. The method of claim 1, wherein the method is capable of diagnosing a patient as being seizure-free or not seizure-free, and/or is capable of identifying an active neural source as a target for surgical intervention.

10. (canceled)

11. The method of claim 1, wherein determining the location and size estimates comprises obtaining recordings from a plurality of electrodes that are compatible with electroencephalography (EEG), stereo-electroencephalography (sEEG), and/or magnetoencephalography (MEG).

12. (canceled)

13. The method of claim 1, wherein the method identifies an active neural source with a mapped percentage of at least about 55%, and/or identifies an active neural source with a center of mass localization error of no more than about 6.0 mm.

14. (canceled)

15. A method for determining probability of post-surgical seizure freedom in a patient, the method comprising:
performing epileptogenic zone localization in the patient using the method of claim 1;
determining average epileptogenic zone location based on center of mass of each epileptogenic zone;
determining distance between the average epileptogenic zone location and the center of mass of each epileptogenic zone (dCOM value); and
comparing the average epileptogenic zones locations from patients post-surgery to the patient to determine the probability of post-surgical seizure freedom in the patient.

16. The method of claim 15, further comprising determining a confidence interval associated with the probability of post-surgical seizure freedom in the patient based on historical data of average epileptogenic zones locations in a plurality of post-surgical patients.

17. (canceled)

18. The method of claim 15, further comprising compiling a plurality of average epileptogenic zone locations from patients who are and are not seizure-free into a database.

19. The method of claim 15, further comprising creating a classification system that outputs a score indicating whether the patient will or will not benefit from surgical intervention.

20. The method of claim 19, wherein the classification system comprises assessing an Engel score and/or an ILAE score.

21. A method for determining probability of post-surgical seizure freedom in a patient undergoing mesial temporal ablation therapy, the method comprising:
performing epileptogenic zone localization in the patient using the method of claim 1;
determining the proportion of the patient's epileptogenic zone location originating in the patient's neocortical anterior temporal lobe; and
comparing the proportion of epileptogenic zone locations comprising neocortical anterior temporal lobe onset from patients post-surgery to the patient to determine probability of post-surgical seizure freedom in the patient.

22. The method of claim 21, further comprising determining a confidence interval associated with the probability of post-surgical seizure freedom in the patient based on historical data of average epileptogenic zones locations in a plurality of post-surgical patients.

23. (canceled)

24. The method of claim 21, further comprising compiling a plurality of average epileptogenic zone locations from patients who are and are not seizure-free into a database.

25. The method of claim 21, further comprising creating a classification system that outputs a score indicating whether the patient will or will not benefit from surgical intervention.

26. The method of claim 25, wherein the classification system comprises assessing an Engel score and/or an ILAE score.
